SEQUENCE LISTING (14 sequences)

SEQ ID NO: 1 - DNA, 1092 bp, Leishmania chagasi
gaaccggccaaggaggagcgtgtcgccacgccagagccggcgccagcaccggccccggctccggtggaggaaccccccatggaggagggtgtccccacgcctgctgcgcctgtaggggaggaccaggaggtagctgccgcccccgcaactgaggagccggcgccgattatgcaggtggaaaatacgaacttcaactgggccgacgacgacatccagttccagattcgtcagccgcagtacgcagcggagtacccggtctctgtgctggagaacctggcccgcaatgtcaccttcaaggtgccggcggaccagaacaccatgacgtccgccatcgaggcgctcgcccgcgagcgccaggcctttgagcagatgaagcgcaaccacgaagacgccatgaagagccgcgaggtagagctgaacgcgcaggaggcgaccctgcaagcccgcgagcgccagctgcatgccaacacggagacgctggcggccgagcgccagcagctgattctccagcagtcgcagttccgtgcccagcaggcccaggctcaggcgcaagctcaggcccaggcccagactcagagccagccgcgcacctcgcaaacgcagtccccggcggcccaggtgtccccgagccccgcgcaggccgcacagccggcgcatgcaccgcagttgcagcaccagatgccgccaccgcatcagcagccccagccccgtagcggcaacgtgtacatgggcggtgggtacggcgcgcaagagtggcagccggaccagcgcacgtggggcaacgagggcgcgatgggtgcccctggtttcgacatgtaccaaggtgcctaccccggctcctacgcccagcgcaactaccatatggctggtggtcaccgtggccagatgcgcagcggcaacctgcagcagcagcagttccctggcaaccgcatgcagccgatgatgccaccgccacagcagtacagccgctcgatggccccgctcggtgcctcccgccagcagcagatggacatgaacttcggcggccgctcgcagcagagctacaacccggcgcccaacaccggctacccgcagcgcagcgctcagtggtaa

SEQ ID NO: 2 - protein, 363 aa, Leishmania chagasi
EPAKEERVATPEPAPAPAPAPVEEPPMEEGVPTPAAPVGEDQEVAAAPATEEPAPIMQVENTNFNWADDDIQFQIRQPQYAAEYPVSVLENLARNVTFKVPADQNTMTSAIEALARERQAFEQMKRNHEDAMKSREVELNAQEATLQARERQLHANTETLAAERQQLILQQSQFRAQQAQAQAQAQAQAQTQSQPRTSQTQSPAAQVSPSPAQAAQPAHAPQLQHQMPPPHQQPQPRSGNVYMGGGYGAQEWQPDQRTWGNEGAMGAPGFDMYQGAYPGSYAQRNYHMAGGHRGQMRSGNLQQQQFPGNRMQPMMPPPQQYSRSMAPLGASRQQQMDMNFGGRSQQSYNPAPNTGYPQRSAQW

SEQ ID NO: 3 - DNA, 1344 bp, Leishmania chagasi
gcggagctggcagcccagttggaggcgacttcggctgcgaagacgtcggtggagcaggaccgcgagagaacgagggcggctctggaggcgcgcgttgcggagctggcgagcaagctggagtcgactgcggctgcgaaggccttggttgagcaggaccgcgagagcacgagggccaccttggaggagcggcttcggattgcggaggcgcgagctgcggaacttgccattgagttggatgccactgctgctgcgaaggcttcgatggaacatgaccgcgagagcacgagggccactttagaggagcggcttcggattgcggaggtgcgcggagcggaactggcaagtcagctagaggccactgctgctgcaaaagcgttgttggagcaggaccgcgagagaacgagggccacgctagagcagcagcttcgcgaatccgaggagcgcgctgcggagctgaaggccgagctggaggccactgctgctgcgaagacgtcggcggagaaggaccgcgagaacacgagggctgctctggaggagaagctaaggggcaccgaggcgcgcgctgcggagctggcggcccgcctaaaggctattgctgcgatgaaggcgtcgatggtgcaggagcgggaaagcgcgagggatgctctggaggaaaagctaaggggctctgaggtgcgcgctgcggagctggctgctcggctaaaggctgctgttgctgcgaagtcgtcggcggagcaggaccgcgagaacacgagggccacgctagagcagcggcttcgcgaatccgaggagcgcgctgcggagctggcgagtcagctggaggccgctgctgctgcgaagtcgtcggcggagcaggaccgcgagaacacgagggctgctctggaggagaagctaagaggctccgaggagcgcgctgcggagctgggaacccgtgtaaaggctagttctgcggcgaaggctttggcggagcaggagcgggacaggataagggctgccctggaggagaagttgcgtgattcggaggcgcgcgctgcggagctgacgaccaagctggaggccactgttgctgcgaagtcgtcggcggagcaggagcgggagaatataaaggtggctttagaggaagaattggttgatgcaagggcgaaattggctggaatggaggcgtcgttgaaggaatcgaagttggagtttgaaggtcgtgtcggggagcttgaaggagagtgcgagaagctgaggaatgataaggtcaggtatgcgaaaaaggttcaatcgcttgagtatcagatgcgcatcgatgaggctcgactgaaggcgcgtcgtgatgcggttcatcgaaaggagtga

SEQ ID NO: 4 - protein, 441 aa, Leishmania chagasi
AELAAQLEATSAAKTSVEQDRERTRAALEARVAELASKLESTAAAKALVEQDRESTRATLEERLRIAEARAAELAIELDATAAAKASMEHDRESTRATLEERLRIAEVRGAELASQLEATAAAKALLEQDRERTRATLEQQLRESEERAAELKAELEATAAAKTSAEKDRENTRAALEEKLRGTEARAAELAARLKAIAAMKASMVQERESARDALEEKLRGSEVRAAELAARLKAAVAAKSSAEQDRENTRATLEQRLRESEERAAELASQLEAAAAAKSSAEQDRENTRAALEEKLRGSEERAAELGTRVKASSAAKALAEQERDRIRAALEEKLRDSEARAAELTTKLEATVAAKSSAEQERENIKVALEEELVDARAKLAGMEASLKESKLEFEGRVGELEGECEKLRNDKVRYAKKVQSLEYQMRIDEARLKARRD

SEQ ID NO: 5 - DNA, 2355 bp, Leishmania chagasi
atggcggacgctgttgggaacacaaactcgaaggtgaagctaaacaagctgatcgtcgaggagccgtacaatgatgacaacagtgttgtatcgctgaacccgaagcggatggaggagctgaacatcttccgcggtgacaccgttctcgtgaagggcaagaaacaccgcagcaccgtctgcatcgcgatggaggacgacgagtgcccgccggagaaaatcaagatgaacaaagtcgcgcgccgcaacatccgcattcacctcggcgacaccattcgcatcgtgccctgcaaggatgttccatacggaaaccgcgttcacctgctgccaatcgatgacacagtggagaacctcactggtgacttgttcgagaacttcttgaagccatactttttggagtcgtaccggcctgtgaagaagggcgactcctttgtctgccgtggcgctatgcgctctgtggaattcaaggtagtggaggtggacccgggtgattactgcatcgtgtcgccggacacaatcatccattccgagggtgatccgatccatcgagaggacgaggaggcgcttgacggagtcggctacgacgacattggtggttgccgcaagcagctgaaccagatccgagagatggtcgagctgcccattcgccacccagagctgttcaagaacattggcatcaagcccccgcgtggcatcttgctttacggccctcctgggagcggcaagacgctcatcgcgcgcgctgtcgcaaatgagacgggcgcctttttcttcctcatcaacggtcctgagattatgagcaagatggctggcgagtcggagagcaaccttcgcaaggccttcgaggaggccgagaagaacgcgccggcgatcatctttatcgatgaaatcgactccatcgccccgaagcgtgagaaagctcagggcgaggtggagaagcgtatcgtttcgcaacttctaaccctaatggacggcatgaaatcgcgctcgcaggtgatcgtgatggcggcgacgaaccgcccgaacaccatcgatccagccttgcgccgttttggccgcttcgaccgcgagctcgacatcggcgtgccggacgagactggccgcttggagatcattcgtatccacaccaagaacatgaagctggccgacgacattgatctggagaaggtggcaaaggactcgcacggctttgtcggcgccgatttggcgcagctctgcaccgaggccgccatgcagtgcattcgcgagaagctgtccatcatcgactgggaggacgacaccatcgatgtcgaggtgatgaacgcgatgtgtgtaacgcaggagcactttcgcgaggccatggcgaagacgaacccgtccgcgctgcgcgagacacaggtggaaacgccgaacgtcgtctgggaggatgtgggcggcctcctcgacgtcaagcgcgagctgcaggagctggtgcagtaccccgtcgagtacccgtggaagtttgagaagtacggcatgtcgccgccgaagggtgtcctcttctacggcccacccggttgcggtaagacgctgctggccaaggcgattgcgacggagtgccaggcgaacttcatctccatcaaaggcccggagcttctgacaatgtggttcggtgaatcggaggcgaatgtgcgcgacgtcttcgacaaggctcgtgctgcggccccttgcgtgctcttttttgatgaactggactccgtggccaagtcccgcggcgcccacggcgacggaggcgcgagcgaccgcgtgatcaaccaaatcctgacagagatggacggcatgaacgtcaagaagaatgtcttcattatcggtgcgacgaaccggccagacgtgctggaccctgcgatcatgcgtcctgggcgtctcgaccagctcatctacatcccattgcccgacaaggcgtctcgtgtggccattatcaaggcgagcttccgtaagtcgccgctagcctccgacgtcgacgtggatcagattgccgccgccacgcacggcttctccggcgccgacttgtctggcatctgccagcgcgcgtgcaagatggcaatccgcgagtcgatcaacaaggaaattcagctcgaggagctgaagaagatcgggcagttggacgaaaacgctgatatcgacccggtgccggagatcacgcgcgctcacgtggaggaggcaatgcgcggtgcccgccgctccgtctctgacgcggacattcggcgctacgatatgttcaagacctccttgcagcagagccgcaccttcggtgctagtaacccgcccccagcggaggccggtgcgcctgctggcagcggggcgccgccgccagcggacgacgacgacctctacagctaa

SEQ ID NO: 6 - protein, 784 aa, Leishmania chagasi
MADAVGNTNSKVKLNKLIVEEPYNDDNSVVSLNPKRMEELNIFRGDTVLVKGKKHRSTVCIAMEDDECPPEKIKMNKVARRNIRIHLGDTIRIVPCKDVPYGNRVHLLPIDDTVENLTGDLFENFLKPYFLESYRPVKKGDSFVCRGAMRSVEFKVVEVDPGDYCIVSPDTIIHSEGDPIHREDEEALDGVGYDDIGGCRKQLNQIREMVELPIRHPELFKNIGIKPPRGILLYGPPGSGKTLIARAVANETGAFFFLINGPEIMSKMAGESESNLRKAFEEAEKNAPAIIFIDEIDSIAPKREKAQGEVEKRIVSQLLTLMDGMKSRSQVIVMAATNRPNTIDPALRRFGRFDRELDIGVPDETGRLEIIRIHTKNMKLADDIDLEKVAKDSHGFVGADLAQLCTEAAMQCIREKLSIIDWEDDTIDVEVMNAMCVTQEHFREAMAKTNPSALRETQVETPNVVWEDVGGLLDVKRELQELVQYPVEYPWKFEKYGMSPPKGVLFYGPPGCGKTLLAKAIATECQANFISIKGPELLTMWFGESEANVRDVFDKARAAAPCVLFFDELDSVAKSRGAHGDGGASDRVINQILTEMDGMNVKKNVFIIGATNRPDVLDPAIMRPGRLDQLIYIPLPDKASRVAIIKASFRKSPLASDVDVDQIAAATHGFSGADLSGICQRACKMAIRESINKEIQLEELKKIGQLDENADIDPVPEITRAHVEEAMRGARRSVSDADIRRYDMFKTSLQQSRTFGASNPPPAEAGAPAGSGAPPPADDDDLYS

SEQ ID NO: 7 - DNA, 1140 bp, Leishmania chagasi
atgtcctcgtcaaataagcagactgtgcgtgtcacttacatctggttgtccggcaaagattcacatcatgatattcgcagcaaggaccgaacgatgtacctgtctcaggagcatgttgcaaagcacccgaaggacttgcttgccaatggcgttttcccggtctggaatttcgacggctcttccactggtcaggcgaaggggctagacactgaaattctgctgaagcctgttaatgcgtttccgtgctgcctcccaaggacgtcgtcaaagattccgtggatcttggtgctcgctgagtgctaccttcctagcggcgagccgacaagagacaattctcgtgccacagctcgcgaaacattcgagcagtgccccgaggagcatccgtggtttggcctggagcaggagtacttcatcatgggacgtgatgggcgcccttatggatggcccgctcacggatttcccgcgccgcagggggcgtactactgcagcaccggctcgaagtcagcgtggggtcgcaagttctgcgaccagcactacgaggtgtgcctcgagatggggctgaacatatcaggcacgaacgcggaggtgactcctggacagtgggagttccagataggcccctgcgaaggcctcgagatgggcgatcagctgactgttgcgcgctgggtgcttttgcgtctgctggaggaagagggtctggatgcggactaccacgcgaaaccgattcagggtgactggaatggaagcggcctgcatacaaacttctcgactgaatcgacccgcgctgaaaacggccttgaggtcattcatcagtatatcgaccgcctcagcaagaccgttagcaaagatatcgtattttatggatccgagaacaacgagcggcttacaggcaagcacgagacgtcgaaggtatctgagttcagcgctggcgtgggtacccgctgtacttcgattcgcatccccaacgcggtcgccagtgagggaaagggatacatggaggatcgccgccctgctggtgatgccgacccgtacttggtgacctcgcgtctttttgcctcgtgcatcggcttggagacaccgtctctcgatctggcgtctccgacacacgagagggactggatgcgtaatgctttcaagtaa

SEQ ID NO: 8 - protein, 379 aa, Leishmania chagasi
MSSSNKQTVRVTYIWLSGKDSHHDIRSKDRTMYLSQEHVAKHPKDLLANGVFPVWNFDGSSTGQAKGLDTEILLKPVNAFPCCLPRTSSKIPWILVLAECYLPSGEPTRDNSRATARETFEQCPEEHPWFGLEQEYFIMGRDGRPYGWPAHGFPAPQGAYYCSTGSKSAWGRKFCDQHYEVCLEMGLNISGTNAEVTPGQWEFQIGPCEGLEMGDQLTVARWVLLRLLEEEGLDADYHAKPIQGDWNGSGLHTNFSTESTRAENGLEVIHQYIDRLSKTVSKDIVFYGSENNERLTGKHETSKVSEFSAGVGTRCTSIRIPNAVASEGKGYMEDRRPAGDADPYLVTSRLFASCIGLETPSLDLASPTHERDWMRNAFK

SEQ ID NO: 9 - DNA, 1473 bp, Leishmania chagasi
tctcaagcctccccgaagaaagtggatagcgcacccaagcagcaacacgcagagccaatgatgacctacaagctcctcgcccccctccatccggagagcgcacgcgcccagaagataatggtggccgccgcgtacgccaacgtcgacgtcgagctcaaggtgtgccagtacggccaggagaacgagacacccgagtttgcgcgcaactgcagcccgtgcatgcgcttcccctccatgcagacggaggagggctacctcttcgagtccaacgccatcatgcgccacatcgcccgtgtggagaagagcggcgcgaagctttacggcgccacacccttcgagagcagccaggtcgacatgtggcttgacttcgcctccagcgagctcgatgcgcacaacatgccgtacctgatggagaccttcgccggcattccggccgcggagagcgccatggcgaccctggaggagagccttgcgggtctggagctgtggctggagacccgcaccttcctcgtcggcgagcgcatgaccgtggccgacatctccgtcgcctttgcgctgcagtgggtgtaccgcatgaacgtgaagcacggcgaggagctgacgaagaagtaccgcaacgcgtaccgcctgtacaacacggtgatgcagcagcccaagacggtggaggtgctgaagcagtggggcgcgacgttcggcccggggaaggcgccgaagaaggccgcggaggcgaagcccaaggcggagaagaagccgaaggaggcggtgggggacgaggaggagcagtcggcgaaggaggagaagaaagcgaacccgctggacgcgctgccgccgagcagcttcgtgctggacgcctacaagcgcgagtacagcaacaccgacacccgcacggtggcggcgccgtacttcttcgagcactacgacacggagggctacacgtgcttctgggcgcgctacaagtacaacgaggataacaagaagcagttcatgacggccaaccttgtgcgcggctggttccagcgcatggagcacgtgcgcaagtacgccttcggcgtcgcgcttatcatcggcgaggacgcggcgcacgagctggtgagcttctgggtgttccgcggcaagggcatgccagagatcgtgagcgaagtggtggacacggagttgttcgagtgggaggagatcaaggacgtgcaggcggagaaggaaagatcacggactacctgtgctgggagggcccgacgatcccgcgcccagtgctggagggtcgctgcttcaagtaagccggccggccgccgccgcctctctcgagcgtgtctcctatcttgtacctctctttttactttgtgttcctttgatcgtcctctcctgttgaccgaaccgggggggacctccacgtgccactctctctcaaacttcggtcacatcccgtggcggacatggaaagagggagggaacgggaggctgtgggtcgtgcgctga

SEQ ID NO: 10 - protein, 490 aa, Leishmania chagasi
SQASPKKVDSAPKQQHAEPMMTYKLLAPLHPESARAQKIMVAAAYANVDVELKVCQYGQENETPEFARNCSPCMRFPSMQTEEGYLFESNAIMRHIARVEKSGAKLYGATPFESSQVDMWLDFASSELDAHNMPYLMETFAGIPAAESAMATLEESLAGLELWLETRTFLVGERMTVADISVAFALQWVYRMNVKHGEELTKKYRNAYRLYNTVMQQPKTVEVLKQWGATFGPGKAPKKAAEAKPKAEKKPKEAVGDEEEQSAKEEKKANPLDALPPSSFVLDAYKREYSNTDTRTVAAPYFFEHYDTEGYTCFWARYKYNEDNKKQFMTANLVRGWFQRMEHVRKYAFGVALIIGEDAAHELVSFWVFRGKGMPEIVSEVVDTELFEWEEIKDVQAEKERSRTTCAGRARRSRAQCWRVAASSKPAGRRRLSRACLLSCTSLFTLCSFDRPLLLTEPGGTSTCHSLSNFGHIPWRTWKEGGNGRLWVVR

SEQ ID NO: 11 - DNA, 768 bp, Leishmania chagasi
caggctgttggcccgctctccgttggcccgcaggcatgttggcccgctctccgttggcccgcaggactgttggcaccgctctccgttggcccgcagctgttggcccgctctctgttggtccgcagtccgttggcccgctctccgttggtaccgcagtccgtcggcccgctctctgttggcccgctctctgttggtccgcagtccgtcggcccgctctctgttggtccgcagtccgttggcccgctctctgttggcccgcagtccgttggcccgctctccgttggcccgcagtccgtcggcccgctctccgttggcccgcaggctgttggcccgctctctgttggtccgcagtccgttggcccgctctctgttggcccgcagtccgtcggtccgctctctgttggtccgcaggctgttggcccgctctccgttggcccgcaggctgttggcccgctctccgttggtccgcaggctgttggcccgctctccgttggcccgcaggctgttggcccgctctccgttggcccgcaggctgttggcccgctctccgttggtccgcaggctgttggcccgctctccgtcggcccgcaggctgttggcccgctctccgttggcccgcaggctgttggcccgctctccgttggcccgcaggctgttggcccgctctccgttggcccgcagtctgtcggcccgctctccgttggtccgcagtccgttggcccgctctcggttggcctgcaggctgttgacgtttctccggtgtcttaa

SEQ ID NO: 12 - protein, 255 aa, Leishmania chagasi
QAVGPLSVGPQACWPALRWPAGLLAPLSVGPQLLARSLLVRSPLARSPLVPQSVGPLSVGPLSVGPQSVGPLSVGPQSVGPLSVGPQSVGPLSVGPQSVGPLSVGPQAVGPLSVGPQSVGPLSVGPQSVGPLSVGPQAVGPLSVGPQAVGPLSVGPQAVGPLSVGPQAVGPLSVGPQAVGPLSVGPQAVGPLSVGPQAVGPLSVGPQAVGPLSVGPQAVGPLSVGPQSVGPLSVGPQSVGPLSVGLQAVDVSPVS

SEQ ID NO: 13 - DNA, 785 bp, Leishmania chagasi
cccatgagctggccaaagtcgaactggcgaaggaccgtgccttcctcgaccctgagccggagggcgtgccactggcggacctcccgctcagcgacgacccggagttcaacgtactggcgaagcagcgtcaggcgctgaagaacaccaggaggggccgcgaccccgaaatgaaggacctggaggagaggatgaacgaccgtgtccacgacatcgcaagggagttcctcagcaagcaccgcggctacctgaacccggagccgcagaatgtacccattgccgacatccccctcaaccgcgacccgatcttccgcgaaatggagaacgagctgttgaaggctatgaaggacccccgcagcaatgcgggcaagattgcagagctgcaggacgacctcaacaaccgcgcagacgacctcgcgaaggacctacggcgcaaggagcttgctaatcaggagcaggagcctctcggcgtgccgctggaagagctgccactcaactacgacccgatcctcaatccactggaacgcaagcgccgcgacatcaagaaaaacccgaagcggaatgccgatgtgctgcgcaacctcgagcgggagatcgccgcgcgcatcgatgacatcgcgcgcgactttctggcgaaggagcgtgctttcctggaccaggaaccggagggggtgcaattggagcgcctgccgctgtcagatgacagggagtttcacgaaatggagagggacctgcgcgcgctgaagaagcaaccagcaaagaacagggacgccatcgaggacctcgag

SEQ ID NO: 14 - protein, 260 aa, Leishmania chagasi
ELAKVELAKDRAFLDPEPEGVPLADLPLSDDPEFNVLAKQRQALKNTRRGRDPEMKDLEERMNDRVHDIAREFLSKHRGYLNPEPQNVPIADIPLNRDPIFREMENELLKAMKDPRSNAGKIAELQDDLNNRADDLAKDLRRKELANQEQEPLGVPLEELPLNYDPILNPLERKRRDIKKNPKRNADVLRNLEREIAARIDDIARDFLAKERAFLDQEPEGVQLERLPLSDDREFHEMERDLRALKKQPAKNRDAIEDLE

(19) United States
(12) Patent Application Publication
(10) Pub. No.: US 2009/0028932 A1
(43) Pub. Date: Jan. 29, 2009

(54) VACCINE FORMULATIONS FOR LEISHMANIA

(76) Inventors: Mary E. Wilson, Iowa City, IA (US); Daniella R.A. Martins, Natal (BR); John E. Donelson, Iowa City, IA (US); Selma M.B. Jeronimo, Natal (BR); Kevin W. Bruhn, Santa Monica, CA (US); Noah A. Craft, Venice, CA (US); Jeffery F. Miller, Santa Monica, CA (US)

Correspondence Address: FULBRIGHT & JAWORSKI L.L.P., 600 CONGRESS AVE., SUITE 2400, AUSTIN, TX, 78701, US

(21) Appl. No.: 11/911,655

(22) PCT Filed: Apr. 13, 2006
(86) PCT No.: PCT/US06/14311

§ 371 Date: Aug. 18, 2008

Related U.S. Application Data
(60) Provisional application No. 60/671,729, filed on Apr. 15, 2005.

(51) Int. Cl. A61K 9/127; A61K 39/00; A61P 31/00

(52) U.S. Cl. 424/450; 424/269.1

(57) ABSTRACT

The present invention involves the use of Leishmania antigens and nucleic acids coding therefor as vaccines. It also provides a method of inducing protective immune responses in a subject through the use of specific delivery systems such as protein and/or DNA vaccines and Listeria monocytogenes vectors.

This application claims benefit of priority to U.S. Provisional Application Ser. No. 60/671,729, filed Apr. 15, 2005, the entire contents of which are hereby incorporated by reference.

The U.S. Government own rights in this invention pursuant to grant numbers R01 AI045540 and R03TW01369, both from the NIH/NIAID.

### BACKGROUND OF THE INVENTION

I. Field of the Invention

The present invention relates generally to the fields of medicine, pathology, microbiology, molecular biology, immunology and infectious disease. More particularly, it concerns methods and compositions to treat, inhibit or prevent Leishmania infections.

II. Description of Background

Members of the genus Leishmania infect many vertebrates, including humans, dogs, and rodents. The life cycles of members of the genus involve a vertebrate host and a vector (a sand fly) that transmits the parasite between vertebrate hosts. In the vector, the parasite develops through several transitional forms and eventually takes on a characteristic morphological form known as the promastigote. The organism reproduces asexually in the vector's gut and transforms to the infectious promastigote. When the vector bites the vertebrate host, promastigotes are inoculated into the vertebrate host skin. The promastigotes enter mononuclear cells of the vertebrate host, most of which are phagocytes, through facilitated phagocytosis. It transforms into a form called the amastigote intracellularly. The amastigote reproduces in the host's cells, and eventually spreads to other cells. The spreading process is not well understood, but it is assumed it may involve rupture of the infected cell and infection of adjacent cells by the parasite. The symptoms and pathology associated with leishmaniasis result from proteins called cytokines that are secreted by the amastigote-infected cell, causing local effects such as ulceration and destructive granulomatous lesions as well as systemic effects such as fever and weight loss, due to the cytokines released from infected cells.

There are many different clinical syndromes “diseases” caused by the Leishmania sp. The different Leishmania sp. cause a characteristic clinical syndrome, although there is considerable overlap in the outcome of infections. In some infections caused by L. major, L. tropica, or L. ethiopica in the Old World, or by L. mexicana or L. amazonensis in the New World, the disease is limited disease to the skin site of the vector's bite. This results in a “cutaneous leishmaniasis” (oriental sore, Jericho boil, Aleppo boil, or Dehli boil) that often heals spontaneously. In disease caused by L. donovani or L. infantum in the Old World or by L. chagasi in the New World, amastigote-laden macrophages spread to organs belonging to the reticuloendothelial system (liver, spleen, bone marrow), resulting in “visceral leishmaniasis” (kala-azar or Dum-Dum fever). (Evidence now suggests that L. infantum and L. chagasi may be the same species of Leishmania causing disease in different geographic locations.) L. braziliensis causes cutaneous ulcers, but can later spread to the mucous membranes of the mouth and nose, resulting in “mucocutaneous leishmaniasis” (espundia or uta). Left untreated, cases of visceral leishmaniasis often result in high rates of mortality. The various types of leishmaniasis occur in Central and South America, the Mediterranean littoral including southern Europe and the Middle East, north and central Africa, India-Nepal-Bangladesh, and other parts of southern and central Asia.

Whereas the tropical and subtropical populations are generally on the front line facing these diseases, the risks of canine and human infection in the Mediterranean basin are often underestimated. Visceral leishmaniasis by Leishmania infantum is largely expanded over the different continents of the Old World, and is present everywhere surrounding the Mediterranean basin, such as in the south of France. Though the parasite present in the south of France appears better adapted to dogs than to humans, the number of human cases of leishmaniasis, currently estimated to be a hundred cases per year, has been growing quickly for 10 years and is further increasing with the number of immunodepressed subjects.

Leishmaniasis is considered to be one of the opportunist diseases of AIDS. Approximately 1500 cases of HIV/Leishmania co-infection are counted in the south of Europe which represents 90% of the reported cases in the world, with Spain being the country the most affected, with approximately 60% of these cases. The cases of co-infection AIDS/leishmaniasis pose a serious public health problem to the extent that the available therapeutics are less effective among persons sick with AIDS as well as any immunodepressed person.

In the Mediterranean region, the domestic dog is the main reservoir of the parasite. Canine leishmaniasis, which is a common pathology of the areas surrounding the Mediterranean, manifests itself in various clinical forms which often lead to the death of the animal. The prevalence of canine leishmaniasis can reach 30% of the canine population in some peripheral urban zones. According to Berrahal et al. (1996), 85% of dogs are PCR (Polymerase Chain Reaction) positive in the endemic zone.

At present, there are no effective immunoprophylactic means against these diseases. The treatment of leishmaniases calls for some available molecules: pentavalent antimony, pentamidine, pyrazolopyrimidines, amphotericin B, aminosidine. Today, a consensus seems to be developing with the combination of antimony salts-pyrazolopyrimidines as the treatment of choice for canine leishmaniasis. Nevertheless, the dogs under treatment remain infectious, in spite of the apparent clinical healing of the animal. As such, the symptomatic improvement is not correlated to significant reduction of the parasitic load and that there is an epidemiological risk even if clinical healing continues. This situation is further complicated by the emergence of chemoresistance phenomena.

The organisms causing leishmaniasis belong to the genus, Leishmania. The Leishmania sp., like other eukaryotic pathogens, are complex organisms with the capacity to alter their antigenic characteristics. In addition, because the different Leishmania species are quite distinct organisms, differing in many of their antigenic and biological characteristics and in the typical disease syndromes they cause, use of single vaccine targeted against all species has proven to be problematic. Because no effective vaccine is currently available to combat these diseases, their control must be done by chemotherapy. Chemotherapy is unfortunately jeopardized by long, toxic and costly treatments accompanied by numerous cases of relapse and by the emergence of chemoresistance phenomena. Thus, it appears evident that the treatment of these parasitic diseases over the long term will depend on the discovery of new therapeutic targets and/or vaccines.

Despite many efforts, there presently is no approved mammalian vaccine for the parasitic infection leishmaniasis. Several have been tried, but none are promising enough for development. Thus, there remains a need for new vaccines for this disease.

### SUMMARY OF THE INVENTION

Thus, in accordance with the present invention, there is provided a vaccine comprising one or more of the antigens selected from the group consisting of the Leishmania chagasi homologue of L. infantum hypothetical protein, and the L. infantum K-39 group protein. The vaccine may further comprise both the Leishmania chagasi homologue of L. infantum hypothetical protein and the L. infantum K-39 group protein. The vaccine may further comprise one more of the antigens selected from the group Leishmania chagasi homologue of L. major transitional ER ATPase, the Leishmania chagasi homologue of L. infantum glutamine synthetase, the Leishmania chagasi homologue of Leishmania infantum EF-1γ, the Leishmania chagasi homologue of Leishmania infantum A2, and Leishmania chagasi Lcr-1. The vaccine may be formulated in a lipid delivery vehicle, such as a liposome. The vaccine may further comprise an adjuvant.

In another embodiment, there is provided a vaccine comprising a first expression cassette comprising a nucleic acid segment encoding for an antigen selected from the group consisting of the Leishmania chagasi homologue of L. infantum hypothetical protein and L. infantum K-39 group protein. The first expression cassette may further comprise a nucleic acid segment encoding both the Leishmania chagasi homologue of L. infantum hypothetical protein and the L. infantum K-39 group protein. The vaccine may also further comprise a second expression cassette, where said first expression cassette comprises a nucleic acid segment encoding the Leishmania chagasi homologue of L. infantum hypothetical protein, and said second expression cassette comprises a nucleic acid segment encoding the L. infantum K-39 group protein. The first expression cassette may further comprise a nucleic acid segment encoding comprising one more of the antigens selected from the group Leishmania chagasi homologue of L. major transitional ER ATPase, the Leishmania chagasi homologue of L. infantum glutamine synthetase, the Leishmania chagasi homologue of Leishmania infantum EF-1γ, the Leishmania chagasi homologue of Leishmania infantum M, and Leishmania chagasi Lcr-1.

The expression cassette may be comprised within a replicable expression construct. The replicable expression construct may be a viral vector or a non-viral vector.

The replicable expression construct may be a Listeria monocytogenes vector. The Listeria monocytogenes vector may be avirulent, and may be ActA. The non-viral vector may be naked DNA, and may be formulated in a lipid delivery vehicle, such as a liposome.

In yet another embodiment, there is provided a method of inducing an immune response in a subject comprising providing to said subject one or more of (a) a vaccine comprising one or more of the antigens selected from the group consisting of the Leishmania chagasi homologue of L. infantum hypothetical protein and the L. infantum K-39 group protein; (b) a vaccine comprising a first expression cassette comprising a nucleic acid segment encoding for an antigen selected from the group consisting of the Leishmania chagasi homologue of L. infantum hypothetical protein and L. infantum K-39 group protein; and/or (c) a vaccine comprising an avirulent Listeria monocytogenes vector comprising a nucleic acid segment encoding for an antigen selected from the group consisting of the Leishmania chagasi homologue of L. infantum hypothetical protein and L. infantum K-39 group protein. The method may comprise administering (a) and (b), (a) and (c), (b) and (c), or (a), (b) and (c) to said subject. The method may comprise two or more administrations of the same vaccine. The vaccine may further comprise one more of the antigens selected from the group Leishmania chagasi homologue of L. major transitional ER ATPase, the Leishmania chagasi homologue of L. infantum glutamine synthetase homolog, the Leishmania chagasi homologue of Leishmania infantum EF-1γ, the Leishmania chagasi homologue of Leishmania infantum A2, and Leishmania chagasi Lcr-1 or a nucleic acid segment coding therefor.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from the detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Immunoblot of bacteria transformed with L. chagasi antigens. FIG. 1 reflects an immunoblot demonstrating the sizes of recombinant proteins. All lanes contain bacterial lysates. Lane A: non-transformed bacteria. Lanes B-G: bacteria transformed with the following cDNAs: 425 (B), 503 (C), 314 (D), 419 (E). Proteins were separated on an 8.5% SDS-polyacrylamide gel and immunoblotted with pooled serum from patients with visceral leishmaniasis. Arrows point to unique bands corresponding to recombinant proteins.

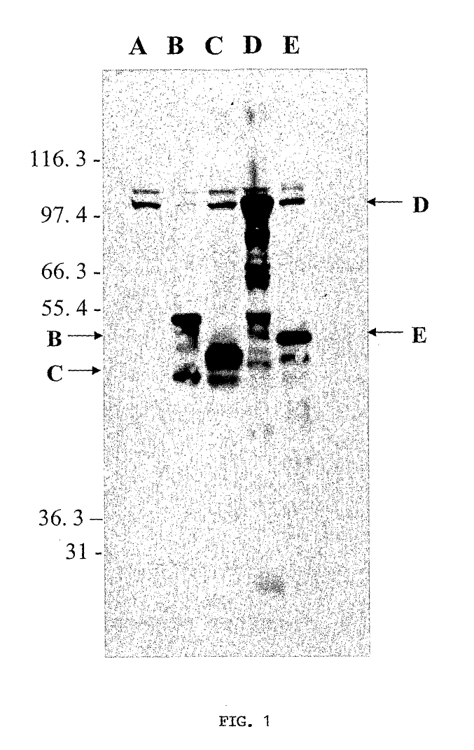

FIG. 2—Use of avirulent L. monocytogenes as an adjuvant during immunization of mice with Leishmania sp. antigens. Mice were immunized with 30 μg of soluble L. chagasi promastigote lysate without or with 10⁷ L. monocytogenes mutants with disrupted genes encoding LLO (hly) or ActA. Control mice received PBS alone. After four weeks, all groups of mice were challenged with 10⁷ live L. chagasi promastigotes through i.v. The total liver parasite load was calculated microscopically on the fourth week of infection with L. chagasi promastigotes.

FIG. 3—Northern blots of total L. chagasi. Wild-type (WT), LcJ promastigote (LcJ) or amastigote (Am) RNA were hybridized with [³²P]-labeled insert DNA from the partial-length of 319, 503, 314, 425, 419, or 648 clones, or with a loading control gene α-tubulin.

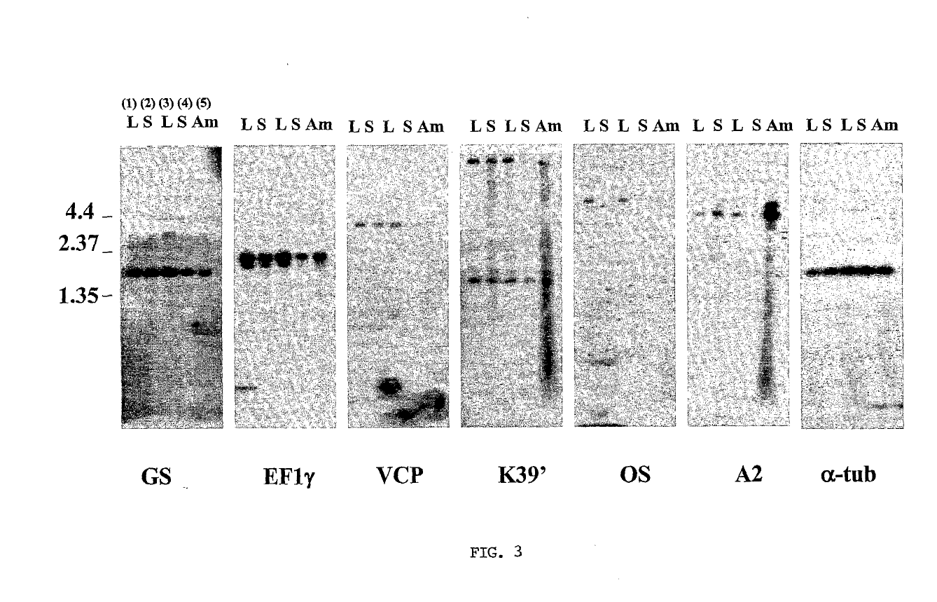

### DESCRIPTION OF ILLUSTRATIVE EMBODIMENTS

The details of the invention are provided below. In one embodiment, the inventors claim a vaccine comprised of several agents selected from an L. chagasi cDNA library—either multiple DNAs for genetic delivery, or multiple proteins corresponding thereto. A particular delivery system for these cDNAs is an engineered avirulent recombinant strain of the bacterium Listeria monocytogenes.

### I. Leishmania Polypeptides or Peptides

In certain aspects, the invention is directed to peptides and polypeptides of Leishmania sp. that can be used to induce a protective immune response in a subject. The invention contemplates specifically where a subject may be a human or an animal such as a companion animal (e.g. dog). The following polypeptides, alone or in combination, are contemplated for use in the invention: Leishmania chagasi homologue of L. infantum hypothetical protein (SEQ ID NO:2), L. infantum K-39 group protein (SEQ ID NO:4), Leishmania chagasi homologue of L. major transitional ER ATPase (SEQ ID NO:6), Leishmania chagasi homologue of L. infantum glutamine synthetase (SEQ ID NO:8), Leishmania chagasi homologue of Leishmania infantum EF-1γ (SEQ ID NO:10), Leishmania chagasi homologue of Leishmania infantum A2 (SEQ ID NO:12), Leishmania chagasi Lcr-1 (SEQ ID NO: 14) or peptide or polypeptide derived therefrom. As used in the context of the present invention, the terms polypeptide and protein are interchangeable.

In certain embodiments, Leishmania peptide or protein compositions of the invention may be provided in the form of natural peptides or proteins isolated from Leishmania organisms. In other embodiments, the protein compositions may be provided by recombinant production using nucleic acids encoding SEQ ID NOS:2, 4, 6, 8, 10, 12, or 14, or polypeptides or peptides derived therefrom. Determination of which peptides or polypeptides, or DNA molecules coding therefore, inhibit Leishmania may be achieved using functional assays that measure Leishmania replication and infectivity, which are familiar to those of skill in the art, for example, Douvas et al. (1985) and Barcinski et al. (1992), both incorporated by reference.

A. Variants of Leishmania Polypeptides

Embodiments of the invention include various Leishmania polypeptides, peptides, and derivatives thereof. Amino acid sequence variants of a peptide or polypeptide can be deletion variants, insertional variants, and/or substitutional variants. Deletion variants lack one or more amino acid residues of the native protein that are not essential for function or immunogenic activity. Insertional variants typically involve the addition of one or more amino acids at a non-terminal point in the polypeptide. This may include the insertion of an amino acid with an immunoreactive epitope or simply a single amino acid residue. Terminal additions, called fusion proteins, are discussed in farther detail below.

Substitutional variants typically contain the exchange of one amino acid for another at one or more sites within the peptide or polypeptide, and may be designed to modulate one or more properties of the peptide or polypeptide, such as stability against proteolytic cleavage, without the loss of other functions or antigenic properties, or alteration of a function that may be toxic to the vaccine delivery vector without loss of antigen properties. In some embodiments, substitutions of this kind are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino as that are identical or biologically functionally equivalent to the amino acids of Leishmania polypeptides or peptides, for example, sequences identical or biologically finally equivalent to SEQ ID NOS:2, 4, 6, 8, 10, 12, or 14, provided the biologically antigenic properties of the protein or peptide is maintained.

The term “functionally equivalent codon” is used herein to refer to codons that encode the same amino acid, such as the six codons for arginine or serine, and also refers to codons that encode biologically equivalent amino acids (see Table 1, below).

Certain embodiments of the invention include various peptides of the Leishmania protein. For example, all or part of a Leishmania protein as set forth in SEQ ID NOS:2, 4, 6, 8, 10, 12, or 14 may be used in various embodiments of the invention. In certain embodiments, a fragment of the Leishmania protein may comprise, but is not limited to about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 550, about 600, about 650, about 700, about 750, about 800, about 850, about 900, about 950 or about 1000 amino acids and any range derivable therein.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological activity (e.g., immunogenicity) where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a Leishmania polypeptide or peptide, to create an equivalent, or even an improved, second-generation molecule. For example, certain amino acids may be substituted for other amino acids in a peptide or protein structure without appreciable loss of interactive binding capacity with structures such as, for example, antigen-binding regions of antibodies, antigen-binding domains of T cell receptors, or binding sites on substrate molecules. Since it is the interactive capacity and nature of a peptide and/or protein that defines that peptide's and/or protein's biological functional activity, certain amino acid substitutions can be made in a peptide and/or protein sequence, and in its underlying DNA or RNA coding sequence, and nevertheless produce a peptide and/or protein with like properties. It is thus contemplated by the inventors that various changes may be made in the DNA sequences of genes or coding regions without appreciable loss of their biological utility or activity, for example, the ability to generate a protective immune response. Table 1 shows the codons that encode particular amino acids.

TABLE 1
CODON TABLE

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making amino acid substitutions, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid substituted for another having a similar hydrophilicity value still produces a biologically equivalent and immunologically equivalent protein.

In certain embodiments, a Leishmania peptide and/or polypeptide may be a fusion protein. Fusion proteins may alter the characteristics of a given polypeptide, such antigenicity or purification characteristics. A fusion protein is a specialized type of insertional variant. This molecule generally has all or a substantial portion of the native molecule, linked at the N- or C-terminus, to all or a portion of a second peptide and/or polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. In some cases, inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals, or transmembrane regions.

B. In Vitro Production of Leishmania Polypeptides and/or Peptides

Various types of expression vectors are known in the art that can be used for the production of proteins and peptides. In addition, different host cells have distinct characteristics and specific mechanisms for the post-translational processing and modification of protein products. Appropriate cell lines or host systems can be chosen to ensure the correct modification and processing of the expressed foreign protein product. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented (for exemplary methods see Freshney, 1992).

If animal cells are used as host cells, the animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

In further aspects of the invention, other protein product production methods known in the art may be used. These methods may include, but are not limited to, use of prokaryotic hosts, yeast hosts, and/or other eukaryotic hosts such as insect cells and the like.

The peptides and/or proteins of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. In certain embodiments, short peptide sequences, or libraries of overlapping peptides, usually from about 6 up to about 35 to 50 amino acids, which correspond to the selected regions described herein, can be readily synthesized and then screened in screening assays designed to identify reactive peptides.

C. Protein Purification

It may be desirable to purify Leishmania peptides and/or proteins, or variants and derivatives thereof. Protein purification techniques are well known to those of skill in the art. These techniques involve, at one level, the crude fractionation of the cellular milieu to polypeptide and non-polypeptide fractions. In addition, one may also choose to separate particular proteins species. Once having generally separated the polypeptide of interest from other proteins, the polypeptide of interest may be further purified using chromatographic and electrophoretic techniques to achieve more complete or complete purification (i.e., purification to homogeneity). Analytical methods particularly suited to the preparation of a pure peptide are ion-exchange chromatography, hydrophobic interaction chromatography, exclusion chromatography; polyacrylamide gel electrophoresis; and/or isoelectric focusing. A particularly efficient method of purifying peptides is fast protein liquid chromatography or FPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally obtainable state. A purified protein or peptide also refers to a protein or peptide free from the environment in which it may naturally occur.

Generally, “purified” will refer to a protein or peptide that has been subjected to fractionation to remove various other components, and which substantially retains its expressed biological activity, for example, immunogenicity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme.

### II. Leishmania Polynucleotides

Certain embodiments of the invention include Leishmania polynucleotides or nucleic acid molecules and fragments thereof. The polynucleotides of the invention may be isolated from Leishmania. The term “isolated” indicates the polynucleotides are free or substantially free from total viral or cellular genomic RNA or DNA, and proteins. It is contemplated that an isolated Leishmania nucleic acid molecule may take the form of RNA or DNA. A Leishmania nucleic acid molecule refers to an RNA or DNA molecule that is capable of yielding all or part of a Leishmania from a transfected cell.

The term “cDNA” is intended to refer to DNA prepared using RNA as a template. The advantage of using a cDNA, as opposed to genomic DNA or an RNA transcript is stability and the ability to manipulate the sequence using recombinant DNA technology (see Maniatis, 1990; Ausubel, 1996). There may be times when the full or partial genomic or cDNA sequence is preferred.

It also is contemplated that a given Leishmania antigen may exist in nature with slightly different nucleic acids, peptides, and polypeptides, or have a slightly different nucleic acid sequence but, nonetheless, encode the same parasite polypeptide or peptide (see Table 1 above) as those of the invention. Consequently, the present invention also encompasses derivatives of Leishmania nucleic acids and/or Leishmania polypeptides and peptides with minimal amino acid changes, wherein the derivative nucleic acids, peptides, and/or polypeptides exhibit the same activities as the non-derivative nucleic acids, peptides, and/or polypeptides.

The term “gene” is used for simplicity to refer to the nucleic acid giving rise to a functional protein, polypeptide, or peptide-encoding unit. As will be understood by those in the art, this functional term includes genomic sequences, cDNA sequences, and smaller engineered nucleic acid segments (a contiguous stretch of nucleobases) that express, or may be adapted to express, proteins, polypeptides, domains, peptides, fusion proteins, and mutants. The nucleic acid molecule encoding a Leishmania antigen may contain a contiguous nucleic acid sequence encoding one or more Leishmania genes and regulatory regions and be of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 10,000 or more nucleotides, nucleosides, or base pairs. Such sequences may be identical or complementary to all or part of SEQ ID NOS: 1, 3, 5, 7, 9, 11, or 13.

In particular embodiments, the invention concerns isolated nucleic acid segments and recombinant vectors with incorporated DNA sequences encoding Leishmania polypeptides or peptides. Such vectors used in the present invention, regardless of the length of the coding sequence itself, may be combined with other DNA or RNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

In a non-limiting example, one or more nucleic acid constructs may be prepared that include a contiguous stretch of nucleotides (i.e., a nucleic acid segment) identical to or complementary to a Leishmania genome. A nucleic acid construct may be about 50, 60, 70, 80, 90, 100, 200, 300, 400, 500, 600, 700, 800, 900, 1,000, 2,000, 3,000, 4,000, 5,000, 6,000, 7,000, 8,000, 9,000, and 10,000, nucleotides in length. Constructs of greater size are contemplated as well, up to and including chromosomal sizes (including all intermediate lengths and intermediate ranges), made possible by the advent of yeast and bacterial artificial chromosomes. It will be readily understood that “intermediate lengths” and “intermediate ranges,” as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.

The nucleic acid segments used in the present invention encompass biologically functional and/or immunogenically equivalent Leishmania proteins and peptides. Such sequences may arise as a consequence of codon redundancy and functional equivalency that are known to occur naturally within nucleic acid sequences and the proteins and peptides thus encoded. Alternatively, functionally and immunologically equivalent proteins or peptides may be created via the application of recombinant DNA technology, in which changes in the peptide or protein structure may be engineered, based on considerations of the properties of the amino acids being exchanged. Changes designed by humans may be introduced through the application of site-directed mutagenesis techniques, e.g., to introduce improvements to the antigenicity of the protein or peptide.

A. Vectors Encoding Leishmania Antigens

The present invention encompasses the use of vectors to encode for all or part of one or more Leishmania peptides and/or proteins. The term “vector” is used to refer to a carrier nucleic acid molecule into which a nucleic acid sequence can be inserted for introduction into a cell where it can be replicated, or to a recombinant microorganism expressing the gene of interest. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), bacteria (e.g., Listeria monocytogenes, Salmonella sp., Mycobacterium bovis BCG), and artificial chromosomes (e.g., YACs, BACs). In particular embodiments, gene therapy or immunization vectors are contemplated. One of skill in the art would be well equipped to construct a vector through standard recombinant techniques, which are described in Maniatis et al (1990), Ausubel et al. (1996), and Current Protocols in Molecular Biology U.S.A.: John Wiley & Sons, Inc. (2000); each incorporated herein by reference.

The term “expression cassette” refers to a nucleic acid segment that contains sufficient information to code for and express a peptide or protein, such as a promoter and a coding region. The term “expression vector” or “expression construct” refers to a vector, including a replicable vector, that contains an expression cassette. In some cases, RNA molecules produced are then translated into a polypeptide or peptide. In other cases, these sequences are not translated, for example, in the production of antisense molecules or ribozymes. Expression cassettes can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra. Thus, a full-length RNA transcript may contain the benefit of recombinant DNA technology such that it contains exogenous control sequences or genes.

1. Promoters and Enhancers

A “promoter” is a control sequence encompassing a region of a nucleic acid sequence at which initiation and rate of transcription are controlled. It may contain genetic elements at which regulatory proteins and molecules such as RNA polymerase and other transcription factors may bind. The phrases “operatively positioned,” “operatively linked,” “under control,” and “under transcriptional control” mean that a promoter is in a correct functional location and/or orientation in relation to a nucleic acid sequence in order to control transcriptional initiation and/or expression of that nucleic acid sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes; promoters or enhancers isolated from any other prokaryotic, viral, or eukaryotic cell; and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed.

In some embodiments, it will be important to employ a promoter and/or enhancer that effectively directs the expression of the nucleic acid segment in the cell type, organelle, and organism chosen for expression. Those of skill in the art of molecular biology generally understand the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or exogenous, i.e., from a different source than the Leishmania sequence. In some examples, a prokaryotic promoter is employed for use with in vitro transcription of a desired sequence. Prokaryotic promoters for use with many commercially available systems include T7, T3, and Sp6.

Table 2 lists several elements/promoters that may be employed, in the context of the present invention, to regulate the expression of a gene. This list is not intended to be exhaustive of all the possible elements involved in the promotion of expression but, merely, to be exemplary thereof. Table 3 provides examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Atchinson et al., 1986, |
| | 1987; Imler et al., 1987; Weinberger et al., |
| | 1984; Kiledjian et al., 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo |
| | et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-DRa | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Omitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion Molecule | |
| (NCAM) | |
| α₁-Antitrypain | Latimer et al., 1990 |
| H2B (TH2B) Histone | Hwang et al., 1990 |
| Mouse and/or | Ripe et al., 1989 |
| Type I Collagen | |
| Glucose-Regulated | Chang et al., 1989 |
| Proteins | |
| (GRP94 and GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh |
| | et al., 1985; Firak et al., 1986; Herr et al., 1986; |
| | Imbra et al., 1986; Kadesch et al., 1986; Wang |
| | et al., 1986; Ondek et al., 1987; Kuhl et al., |
| | 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Chol et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987; Glue et al., 1988 |
| Hepatitis B Virus | Bulla et al., 1986; Jameel et al., 1986; Shaul et |
| | al., 1987; Spandau et al., 1988; Vannice et al., |
| | 1988 |
| Human | Muesing et al., 1987; Hauber et al., 1988; |
| Immunodeficiency | Jakobovits et al., 1988; Feng et al., 1988; |
| Virus | Takebe et al., 1988; Rosen et al., 1988; |
| | Berkhout et al., 1989; Laspia et al., 1989; Sharp |
| | et al., 1989; Braddock et al., 1989 |
| Cytomegalovirus | Weber et al., 1984; Boshart et al., 1985; |
| (CMV) | Foecking et al., 1986 |
| Gibbon Ape | Holbrook et al., 1987; Quinn et al., 1989 |
| Leukemia Virus | |
| L. monocytogenes | Jensen et al., 1997 Immunol Rev 1997; 158: 147- |
| hly | 57. |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; Searle |
| | | et al., 1985; Stuart et al., |
| | | 1985; Imagawa et al., 1987, |
| | | Karin et al., 1987; Angel et |
| | | al., 1987b; McNeall et al., |
| | | 1989 |
| MMTV (mouse | Glucocorticoids | Huang et al., 1981; Lee et |
| mammary | | al., 1981; Majors et al., |
| tumor virus) | | 1983; Chandler et al., 1983; |
| | | Lee et al., 1984; Ponta et al., |
| | | 1985; Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macro- | IL-6 | Kunz et al., 1989 |
| globulin | | |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid | Thyroid Hormone | Chatterjee et al., 1989 |
| Stimulating | | |
| Hormone | | |
| α Gene | | |
| Tetracycline | Tetracycline | [ |
| operator | | |

The identity of tissue-specific promoters or elements, as well as assays to characterize their activity, is well known to those of skill in the art. Examples of such tissue-specific regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse α2 (XI) collagen (Tsumaki et al., 1998), D1A dopamine receptor gene (Lee et al., 1997), insulin-like growth factor II (Wu et al., 1997), and human platelet endothelial cell adhesion molecule-1 (Almendro et al., 1996).

2. Initiation Signals and Internal Ribosome Binding Sites

In some cases, a specific initiation signal may be required for efficient translation of coding sequences. These initiation signals may include the ATG initiation codon or adjacent sequences. Exogenous translational control signals, including the ATG initiation codon, may need to be provided. The exogenous translational control signals and initiation codons can be either natural or synthetic. One of ordinary skill in the art would readily be capable of determining and providing the necessary signals. It is well known that the initiation codon must be “in-frame” with the reading frame of the desired coding sequence to ensure translation of the entire insert. The efficiency of expression of the coding sequence may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well as an IRES element from a mammalian message (Macejak and Sarnow, 1991). IRES elements can be linked to heterologous open reading frames. Multiple open reading frames can be transcribed together, each separated by an IRES element, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include one or more multiple cloning sites (MCS), which are nucleic acid regions that contain multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available and use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments, a termination signal that ends the production of an RNA transcript is contemplated. A terminator may be necessary iii vivo to achieve desirable message levels.

In eukaryotic systems, the terminator region may comprise specific DNA sequences that permit site-specific cleavage of the new transcript to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in embodiments involving eukaryotes, the terminator may comprise a signal for the cleavage of the RNA, and the terminator signal may promote polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be simply a lack of transcribable or translatable sequence, such as due to sequence truncation.

Translation termination signals, e.g., stop codons, can be engineered into any sequence for expression.

5. Polyadenylation Signals

For expression, particularly eukaryotic expression, one will typically include a polyadenylation signal to effect proper polyadenylation of the transcript. As the nature of the polyadenylation signal is not believed to be crucial to the successful practice of the invention, any such sequence may be employed. Certain embodiments include the SV40 polyadenylation signal and/or the bovine growth hormone polyadenylation signal, both of which are convenient and known to function well in various target cells. Along with increasing the stability of the transcript, polyadenylation may facilitate cytoplasmic transport.

6. Origins of Replication

In order to propagate a (plasmid) vector in a host cell, the vector may contain one or more origins of replication sites (often termed “ori”). Ori sites are a specific nucleic acid sequence at which replication is initiated. Alternatively to ori sites, an autonomously replicating sequence (ARS) can be employed if the host cell is yeast.

7. Selectable and Screenable Markers

In certain embodiments of the invention, a marker in the expression vector will be used to identify, either in vitro or in vivo, the cells containing a nucleic acid construct of the present invention. Such markers confer an identifiable change, thus permitting easy identification of cells containing the expression vector. Generally, a selectable marker is one that confers a property that allows for selection. A positive selectable marker is one in which the presence of the marker allows for its selection, while a negative selectable marker is one in which the presence of the marker prevents its selection. An example of a positive selectable marker is a drug resistance marker.

The inclusion of a drug selection marker aids in the cloning and identification of transformants. For example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers, including screenable markers such as GFP, whose basis is colorimetric analysis, are also contemplated. In certain embodiments, screenable enzymes such as herpes simplex virus thymidine kinase (tk) or chloramphenicol acetyltransferase (CAT) may be utilized as markers. Moreover, one of skill in the art would know how to employ immunologic markers, possibly in conjunction with FACS analysis. Further examples of selectable and screenable markers are well known to one of skill in the art. The marker used is not believed to be important, so long as the marker is capable of being expressed simultaneously with the nucleic acid encoding a gene product.

B. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, as well as any transformable organism capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector, expression of part or all of the vector-encoded nucleic acid sequences, or production of infectious viral particles. Numerous cell lines and cultures are available for use as a host cell, and they can be obtained through the American Type Culture Collection (ATCC), an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses. Both virulent and avirulent strains of Listeria monocytogenes have been used as host cells for recombinant plasmid constructs, including those proposed in this invention.

Examples of eukaryotic host cells for replication and/or expression of a vector include, but are not limited to, HeLa, NIH3T3, Jurkat, 293, Cos, CHO, Saos, and PC12 cells. Many host cells from various cell types and organisms are available and would be known to one of skill in the art. A viral vector may be used in conjunction with either a eukaryotic or prokaryotic host cell, particularly with host cells permissive for replication or expression of the vector.

In the present invention, it is contemplated that a source of Leishmania proteins will be host cells capable of producing large amounts of recombinant protein, either in non-glycosylated or glycosylated form. Some choices of host cells are made because the host cell can infect the same mononuclear phagocytes (e.g., macrophages, dendritic cells) as the Leishmania parasite itself. This is the case with the host cell Listeria monocytogenes. In other cases, E. coli will be the host cell because of the ease of producing large amounts of protein. Eukaryotic host cells specific for the vectors named above (e.g., baculovirus, vaccinia virus) may also be used for the purpose of producing recombinant protein.

C. Expression Systems

Numerous expression systems exist that comprise at least all or part of the compositions discussed above. Prokaryote- and/or eukaryote-based expression systems can be employed for use with the present invention to produce nucleic acid sequences, or their cognate polypeptides and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference. The insect cell/baculovirus system can be bought, for example, under the name MAXBAC® 2.0 from INVITROGEN® and BACPACK™ BACULOVIRUS EXPRESSION SYSTEM from CLONTECH®.

Other examples of expression systems include STRATAGENE®'s COMPLETE CONTROL™ Inducible Mammalian Expression System, which involves a synthetic ecdysone-inducible receptor, or STRATAGENE®'s pET Expression System, an E. coli expression system. Another example of an inducible expression system, available from INVITROGEN®, is the T-REX™ (tetracycline-regulated expression) System, an inducible mammalian expression system that uses the full-length CMV promoter. The Tet-On™ and Tet-Off™ systems from CLONTECH® also can be used to regulate expression in a mammalian host using tetracycline or its derivatives. The implementation of exemplary expression systems is described in Gossen et al. (1992; 1995), and U.S. Pat. No. 5,650,298, all of which are incorporated by reference.

INVITROGEN® also provides a yeast expression system called the Pichia methanolica Expression System, which is designed for high-level production of recombinant proteins in the methylotrophic yeast Pichia methanolica. Generally, using an expression system, one of skill in the art would know how to express a vector, such as an expression construct, to produce a nucleic acid sequence or its cognate polypeptide or peptide.

D. Introduction of Nucleic Acids into Cells

In certain embodiments, a nucleic acid may be introduced into a cell in vitro for production of polypeptides and/or peptides or in vivo for immunization purposes. There are a number of ways in which nucleic acid molecules such as expression vectors may be introduced into cells. In certain embodiments of the invention, the expression vector comprises a shuttle vector that can replicate in both E. coli and Listeria monocytogenes. As described in Hsieh et al. (1993), herein incorporated by reference, Listeria monocytogenes is a faculatative anaerobe that induces potent type 1 response in CD4⁺ and CD8⁺ T cells. As such, avirulent gene knockout lines of Listeria monocytogenes expressing foreign proteins, as described in Angelakopoulos et al. (2002) and Starks et al. (2004), herein incorporated by reference, have been developed as vaccine vehicles for malignant and viral diseases. Furthermore, as shown by Reed and Campos-Neto (2003), herein incorporated by reference, immunization of mice with recombinant LACK-expressing Listeria monocytogenes provided protection from or delay of Leishmania major lesion development, although not against parasite growth. Soussi et al. (2002), herein incorporated by reference, illustrate the use of L. monocytogenes expressing LACK to immunize against L. major infection in mice.

As a live vaccine delivery vector, L. monocytogenes is highly immunostimulatory, efficiently enters the endosomal and cytoplasm compartments of APCs generating both CD4⁺ and CD8⁺ responses, is easily cultivated, and can be delivered to humans orally (Kaufmann, 1993). The defined life cycle of L. monocytogenes has allowed avirulent mutants that lack genes essential for survival in a mammalian host to be raised (Harding, 1995; Michel et al., 1990). ActA⁻ or LLO-L. monocytogenes mutants elicit CD4⁺ and CD8⁺ responses and efficiently deliver foreign antigens (Berche et al, 1987; Starks et al, 2004). Protective immunity to recombinant epitopes seems not to be compromised by the use of attenuated L. monocytogenes, or by the pre-existence of immunity to L. monocytogenes in the recipient host. Furthermore, the vaccine vector itself can be killed with antibiotics after administration without compromising immune response development (Starks et al., 2004) making it feasible to consider attenuated L. monocytogenes lines for immunization of populations that might include compromised hosts.

Experimental animals have been successfully immunized with L. monocytogenes expressing β-galactosidase, LCMV, influenza virus nucleoprotein, tumor antigens, and rabbit papillomavirus (Pan et al., 1999; Ikonomidis et al, 1997; Shen et al., 1995; Goossens et al., 1995; Schafer et al., 1992; Jensen et al., 1997; Pan et al., 1995a; Pan et al., 1995b). Immunization of susceptible or resistant strains of mice with L. monocytogenes expressing leishmania LACK resulted in a Th1 response and diminished or delayed L. major lesions, respectively. Avirulent Listeria monocytogenes gene knockout mutants lacking the actA and inlB genes have been engineered for use in vaccination of humans against tumor-associated antigens (Brockstedt et al., 2004). Even more importantly, an avirulent gene knockout L. monocytogenes vaccine strain has already been shown to be safe for use in humans in phase I trials (Angelakopoulos et al., 2002).

In certain embodiments, an expression vector known to one of skill in the art may be used to express a segment of a Leishmania nucleic acid, which may be translated into a Leishmania polypeptide or peptide. The ability of certain viruses to enter cells via receptor-mediated endocytosis, to integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986).

“Viral expression vector” is meant to include those vectors containing sequences of a virus sufficient to (a) support packaging of the vector and (b) to express a Leishmania polynucleotide that has been cloned therein. In this context, expression may require that the gene product be synthesized. A number of such viral vectors have already been thoroughly researched, including adenovirus, adeno-associated viruses, retroviruses, herpesviruses, and vaccinia viruses. One mechanism for delivery of foreign genes is via viral infection where the expression vector is encapsidated in an infectious viral particle.

Several non-viral methods for the transfer of expression vectors into cultured mammalian or non-mammalian cells also are contemplated by the present invention. These non-viral methods include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), liposome (Ghosh and Bachhawat, 1991; Kaneda et al., 1989) and receptor-mediated transfection (Wu and Wu, 1987; Wu and Wu, 1988). Some of these techniques may be successfully adapted for either in vivo or ex vivo use.

Transfer of a nucleic acid molecule may be performed by any of the methods mentioned above through physically or chemically penneabilizing the cell membrane. In certain embodiments, these methods are particularly applicable for transfer for protein production in vitro, but they may be applied to in vivo applications as well.

### III. Pharmaceutical Compositions and Routes of Administration

Pharmaceutical compositions including Leishmania peptides, polypeptides and polynucleotides will be formulated along the line of typical pharmaceutical drug and biological preparations. In many embodiments, the pharmaceutical composition will be in the form of a vaccine. A discussion of formulations may be found in Remington's Pharmaceutical Sciences (1990). The percentage of active compound in any pharmaceutical composition or preparation is dependent upon the activity of the compound, for example the ability of Leishmania vaccines to stimulate an immune response against Leishmania infection. In many embodiments, the pharmaceutical compositions should contain at least 0.1% active compound. The percentage of the active compound in the compositions and preparations may, of course, be varied and may conveniently be between about 2% to about 60% of the weight of the unit. The amount of active compound(s) in therapeutically useful compositions and preparations is generally such that a suitable dosage for treatment will be obtained.

The phrases “pharmaceutically acceptable” or “pharmacologically acceptable” refer to molecular entities and compositions and preparations that do not produce an adverse, allergic, or other untoward reaction when administered to a subject, such as an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions and preparations is contemplated. Supplementary active ingredients, such as other anti-paristical agents, can also be incorporated into the compositions.

Pharmaceutically acceptable salts may be a component of the pharmaceutical compositions and preparations and include the acid salts and those which are formed with inorganic acids such as, for example, hydrochloric or phosphoric acids, or such organic acids as acetic, oxalic, tartaric, mandelic, and the like. Salts formed with free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Various delivery systems are known and can be used to administer the pharmaceutical compositions and preparations of the invention. The pharmaceutical compositions or preparations of the invention and their pharmaceutical acceptable salts may be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For example, if the pharmaceutical composition of the invention is a vaccine, methods of administering a vaccine include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, a vaccine of the invention is administered intramuscularly, intravenously, or subcutaneously. The vaccine of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical composition or preparation of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the pharmaceutical composition or preparation of the invention can be delivered in a controlled release or sustained release system. In one embodiment, a pump may be used to achieve controlled or sustained release. In another embodiment, polymeric materials can be used to achieve controlled or sustained release of the pharmaceutical composition of the invention (see e.g., Medical Applications of Controlled Release, 1974); Controlled Drug Bioavailability, Drug Product Design and Performance, 1984; Ranger and Peppas, 1983; see also Levy et al., 1985; During et al., 1989; Howard et al., 1989; U.S. Pat. Nos. 5,679,377; 5,916,597; 5,912,015; 5,989,463; 5,128,326; International Publication Nos. WO 99/15154 and WO 99/20253, all of which are hereby incorporated by reference. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, 1984).

Controlled release systems are discussed in a review by Langer (1990). Any technique known to one of skill in the art can be used to produce sustained release formulations comprising one or more pharmaceutical compositions or preparations of the invention. See, e.g., U.S. Pat. No. 4,526,938; International Publication Nos. WO 91/05548 and WO 96/20698; Ning et al., 1996; Song et al., 1995; Cleek et al., 1997; and Lam et al., 1997, each of which is incorporated herein by reference in its entirety.

For oral administration, the pharmaceutical compositions or preparations may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the pharmaceutical compositions or preparations of the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The pharmaceutical compositions of the invention may also be formulated in rectal compositions such as suppositories or retention enemas, e.g., containing conventional suppository bases such as cocoa butter or other glycerides.

In addition to the formulations described previously, the pharmaceutical compositions or preparations may also be formulated as a depot preparation. Such long acting formulations may be administered by implantation (for example subcutaneously or intramuscularly) or by intramuscular injection. Thus, for example, the pharmaceutical compositions or preparations may be formulated with suitable polymeric or hydrophobic materials (for example as an emulsion in an acceptable oil) or ion exchange resins, or as sparingly soluble derivatives, for example, as a sparingly soluble salt.

The invention also provides that a pharmaceutical composition or preparation of the invention is packaged in a hermetically sealed container such as an ampoule or sachette indicating the quantity. In one embodiment, where the pharmaceutical composition or preparation is a vaccine, the vaccine is supplied as a dry sterilized lyophilized powder or water free concentrate in a hermetically sealed container and can be reconstituted, e.g., with water or saline to the appropriate concentration for administration to a subject.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

As stated above, the pharmaceutical compositions and preparations of the present invention can be formulated for parenteral administration, e.g., formulated for injection via the intradermal, intravenous, intramuscular, intrathoracic, sub-cutaneous, and/or intraperitoneal routes. Administration of the pharmaceutical compositions and preparations through intradermal and intramuscular routes is specifically contemplated. The pharmaceutical compositions and preparations may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Pharmaceutical compositions and preparations suitable for injectable use include sterile aqueous solutions or dispersions and sterile powders for the extemporaneous preparation of sterile injectable solutions or dispersions. In most embodiments suitable for injectable use, the composition or preparation must be sterile and must be fluid to the extent that easy injection is possible. It should generally be stable under the conditions of manufacture and storage and be preserved against the contaminating action of microorganisms, such as bacteria and fingi. A carrier used with injectible forms of the pharmaceutical compositions and preparations can be a solvent or dispersion medium containing, for example, water, ethanol, polyol (for example, glycerol, propylene glycol, and liquid polyethylene glycol, and the like), suitable mixtures thereof, and vegetable oils. The proper fluidity of the injectible pharmaceutical composition or preparation can be maintained, for example, by the use of a coating, such as lecithin, by the maintenance of required particle size in the case of dispersion, and by the use of surfactants. The prevention of the contaminating action of microorganisms can be brought about by various antibacterial and antifungal agents, for example, parabens, chlorobutanol, phenol, sorbic acid, thimerosal, phenylmercuric nitrate, m-cresol, and the like. In many embodiments, it will be preferable to use isotonic solutions as carriers, for example, sugars or sodium chloride. If desired, prolonged absorption of the injectable compositions or preparations can be brought about by the use in the compositions of agents that delay absorption, for example, aluminum monostearate, and gelatin.

In some embodiments, sterile injectable solutions are prepared by incorporating the active compound(s) in the required amount in the appropriate solvent with various of the other ingredients enumerated above, as required, followed by sterile filtration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques, which yield a powder of the active ingredient plus any additional desired ingredient.

For parenteral administration of an aqueous solution, the solution may be suitably buffered, if necessary, such as by first rendering the liquid diluent isotonic with sufficient saline or glucose. These particular aqueous solutions are especially suitable for intravenous, intramuscular, subcutaneous, intradermal, and intraperitoneal administration. For exampleterile aqueous media that can be employed as buffering agents will be known to those of skill in the art in light of the present disclosure. For example, one dosage of the pharmaceutical composition or preparation could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the age and possibly medical condition of the subject being treated. The person responsible for administration will, in any event, determine the appropriate dose for the individual subject.

An effective amount of the pharmaceutical composition or preparation is determined based on the intended goal. The term “unit dose” or “dosage” refers to physically discrete units suitable for use in a subject, each unit containing a predetermined quantity of the pharmaceutical composition or preparation calculated to produce the desired responses, in association with its administration, i.e., the appropriate route and treatment regimen. The quantity to be administered, both according to number of treatments and unit dose, depends on the protection desired.

When included in the pharmaceutical compositions or preparations of the invention, peptides or polypeptides may be administered in a dose that can vary from 0.01, 0.05, 0.1, 0.5, 1, 5, 10, 20, 30, 40, 50, 60, 70, 80, 90, 100 mg/kg of weight to 50, 60, 70, 80, 90, 100, 110, 120, 130, 140, 150, 160, 170, 180, 190, 200 mg/kg of weight in one or more daily, weekly, monthly, or yearly administrations during one or various days, weeks, months, or years. For a vaccine, the goal will be to develop a formulation that elicits protective immunity in as few doses as possible, hopefully a single dose. It is possible that booster doses will be required either for the primary immunization or for repeated immunization as the initial immune response wanes. As stated above, the antigens or genes encoding antigens of the invention can be administered by parenteral injection (intravenous, intraperitoneal, intramuscular, subcutaneous, intracavity, intradermal or transdermic). If the pharmaceutical compositions or preparations comprise viral vectors as the host delivery system, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient. Similar figures may be extrapolated for bacterial host delivery systems, or for liposomal or other non-viral host delivery systems, by comparing relative uptake efficiencies. Formulation of the delivery system as a pharmaceutically acceptable composition is discussed below.

In many instances, when the pharmaceutical composition is a vaccine, it will be desirable to have several or multiple administrations of the vaccine. As a vaccine, the pharmaceutical compositions and preparations of the invention may be administered 1, 2, 3, 4, 5, 6, 7, 8, 9, 10, or more times. The administrations will normally be at from one week to twelve week intervals. In certain embodiments, the administrations will be from one week to four week intervals. Periodic re-administration of vaccine may be desirable with recurrent exposure to the pathogen.

Protein vaccines often employ an immunogenic carrier protein, such as hepatitis B surface antigen, keyhole limpet hemocyanin or bovine serum albumin. Methods for including these carrier proteins in vaccines are well known in the art. Other immunopotentiating compounds such as polysaccharides, including chitosan, which is described in U.S. Pat. No. 5,980,912, hereby incorporated by reference, are also contemplated for use with the compositions of the invention. The vaccine may further comprise an adjuvant, such as alum, Bacillus Calmette-Guerin, agonists and modifiers of adhesion molecules, tetanus toxoid, imiquinod, montanide, MPL, and QS21.

In a particular embodiment of the invention, the Leishmania vaccine may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991).

Current in vivo lipid delivery methods use subcutaneous, intradermal, intratumoral, or intracranial injection to avoid the toxicity and stability problems associated with cationic lipids in the circulation. The DOTAP:cholesterol lipid formulation is said to form a unique structure termed a “sandwich liposome.” This formulation is reported to “sandwich” DNA between an invaginated bi-layer or ‘vase’ structure. Beneficial characteristics of these lipid structures include a positive ρ, colloidal stabilization by cholesterol, two-dimensional DNA packing and increased serum stability.

The production of lipid formulations often is accomplished by sonication or serial extrusion of liposomal mixtures after (I) reverse phase evaporation, (II) dehydration-rehydration, (III) detergent dialysis, and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990).

### IV. EXAMPLE

The following example is included to demonstrate preferred embodiments of the invention. It should be appreciated by those of skill in the art that the techniques disclosed in the example which follows represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The inventors systematically screened an L. chagasi amastigote cDNA library for antigens that could be protective. The library was first immunoscreened using pooled serum from Brazilians with visceral leishmaniasis, yielding 242 protein-producing clones. Each positive phage clone was induced with IPTG and re-screened for its ability to cause proliferation of immune T cells from C3H.HeJ mice infected with L. chagasi. These mice are genetically resistant to L. chagasi, and they are hypo-responsive to LPS, which could contaminate recombinant proteins. After the exclusion of heat shock proteins and proteins of small size, six unique clones were identified. Their physical characteristics are listed in Table 4.

TABLE 4
Leishmania chagasi antigens identified with the double screen. The
size and physical characteristics of each Leishmania chagasi cDNA
clone, both predicted and observed, is shown in the table below.

| | Insert | ORF | ORF | Predicted | Observed | | |
|---|---|---|---|---|---|---|---|
| | Size | Size | Size | Protein | Protein Mass | Homologues | SEQ ID |
| Clone | (bp) | (bp) | (aa) | Mass | (Immunoblot) | (% identity *) | NO: |
| 314 | 3076 | 2355 bp | 785 aa | 86.8 kDa | 98 kDa | L. major | 5 and 6 |
| | | | | | | transitional ER | |
| | | | | | | ATPase (99%) | |
| 319 | 1770 | 1140 bp | 380 aa | 42.4 kDa | NV ** | L. infantum | 7 and 8 |
| | | | | | | glutamine | |
| | | | | | | synthetase | |
| | | | | | | (100%) | |
| 419 | 2073 | 1092 bp | 364 aa | 40.1 kDa | 53 kDa | L. infantum | 1 and 2 |
| | | | | | | hypothetical | |
| | | | | | | protein (100%) | |
| 425 | 1644 | 1344 bp | 448 aa | 49.2 kDa | 60 kDa | L. infantum K- | 3 and 4 |
| | | | | | | 39 (88%) | |
| 503 | 2009 | 1473 bp | 491 aa | 55.6 kDa | 50 kDa | L. infantum | 9 and 10 |
| | | | | | | EF-1γ(100%) | |
| 648 | 2886 | 768 bp | 256 aa | 23.9 kDa | NV ** | L. infantum A2 | 11 and 12 |
| | | | | | | (97%) | |

* Percent amino acid identity with sequences from GenBank or the Leishmania major and L. infantum GeneDB genomic databases at the Sanger Centre. GenBank accession numbers of homologues are: clone 314 (transitional ER ATPase): CAJ09090; clone 319 (glutamine synthetase): CAJ02080; clone 419 (L. infantum hypothetical protein): CAJ02805; clone 425 (kinesin-like protein): AAA29254; clone 503: identical to L. infantum EF-1γ accession number CAC35543; clone 648 (A2 gene class member): AAB30592.
** NV - Bands corresponding to recombinant proteins were not visualized on immunoblots.

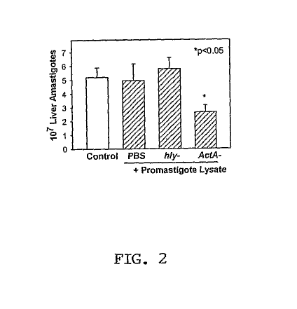

The inventors further found that live L. monocytogenes mutants lacking the actA gene were effective adjuvants during L. chagasi immunization, whereas hly-deficient mutants were not (FIG. 2). Although not meant to be limiting, the hly-L. monocytogenes may have failed because the mutants do not enter the host cell cytoplasm and stimulate CD8⁺ responses, or because of the absence of the highly antigenic LLO protein (Sanderson et al., 1995; Safley et al., 1991). When non-recombinant L. monocytogenes are used as adjuvant with soluble antigen, ActA-L. monocytogenes mutants express LLO and are potent inducers of both CD4⁺ and CD8⁺ responses (Starks et al., 2004). The L. monocytogenes expressing the recombinant antigens themselves may be more efficient in inducing immunity than the killed system illustrated in FIG. 2, because the live L. monocytogenes may deliver the antigen to the exact intracellular location where the L. monocytogenes delivery host will be processed to stimulate immune responses, and where the intracellular Leishmania parasite resides.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,526,938
U.S. Pat. No. 4,554,101
U.S. Pat. No. 4,683,202
U.S. Pat. No. 4,879,236
U.S. Pat. No. 5,128,326
U.S. Pat. No. 5,650,298
U.S. Pat. No. 5,679,377
U.S. Pat. No. 5,871,986
U.S. Pat. No. 5,912,015
U.S. Pat. No. 5,916,597
U.S. Pat. No. 5,925,565
U.S. Pat. No. 5,928,906
U.S. Pat. No. 5,935,819
U.S. Pat. No. 5,980,912
U.S. Pat. No. 5,989,463
Almendro et al., J. Immunol., 157(12):5411-5421, 1996.
Angel et al., Cell, 49:729, 1987b.
Angel et al., Mol. Cell. Biol., 7:2256, 1987a.
Angelakopoulos et al., Infect. Immun., 70:3592-3601, 2002.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Banerji et al., Cell, 27(2 Pt 1):299-308, 1981.
Banerji et al., Cell, 33(3):729-740, 1983.
Barany and Merrifield, In: The Peptides, Gross and Meienhofer (Eds.), Academic Press, NY, 1-284, 1979.
Barcinski et al., Infect. Immun., 60:3523-3527, 1992.
Berche et al., J. Immunol., 138:2266-2271, 1987.
Berkhout et al., Cell, 59:273-282, 1989.
Berrahal et al., Am. J. Trop. Med. Hyg., 55:273-277, 1996.
Blanar et al, EMBO J, 8:1139, 1989.
Bodine and Ley, EMBO J, 6:2997, 1987.
Boshart et al., Cell, 41:521, 1985.
Bosze et al., EMBO J., 5(7):1615-1623, 1986.
Braddock et al., Cell, 58:269, 1989.
Brockstedt et al., Proc. Natl. Acad. Sci. USA, 101(38):13832-37, 2004.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Celander and Haseltine, J. Virology, 61:269, 1987.
Celander et al., J Virology, 62:1314, 1988.
Chandler et al., Cell, 33:489, 1983.
Chang et al., Mol. Cell. Biol., 9:2153, 1989.
Chatterjee et al., Proc. Natl. Acad. Sci. USA, 86:9114, 1989.
Chen and Okayama, Mol. Cell. Biol., 7(8):2745-2752, 1987.
Choi et al., Cell, 53:519, 1988.
Cleek et al., Pro. Int'l. Symp. Control. Rel. Bioact. Mater., 24:853-854, 1997.
Cocea, Biotechniques, 23(5):814-816, 1997.
Cohen et al., J. Cell. Physiol., 5:75, 1987.
Controlled Drug Bioavailability, Drug Product Design and Performance, Smolen and Ball (Eds.), Wiley, NY, 1984.
Costa et al., Mol. Cell. Biol., 8:81, 1988.
Cripe et al., EMBO J, 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Douvas et al., Infect. Immun., 50:1-8, 1985.
During et al., Ann. Neurol., 25:351, 1989.
Edbrooke et al., Mol. Cell. Biol., 9:1908, 1989.
Edlund et al., Science, 230:912-916, 1985.
Fechheimer, et al., Proc Natl. Acad. Sci. USA, 84:8463-8467, 1987.
Feng and Holland, Nature, 334:6178, 1988.
Firak and Subramanian, Mol. Cell. Biol., 6:3667, 1986.
Flesch and Kaufmann, Res. Immunol., 146:520-525, 1995.
Foecking and Hofstetter, Gene, 45(1):101-105, 1986.
Fraley et al., Proc. Natl. Acad. Sci. USA, 76:3348-3352, 1979.
Freshney, In: Animal Cell Culture, A Practical Approach, 2^{nd} Ed., Oxford Press, UK, 1992.
Fujita et al., Cell, 49:357, 1987.
Gabizon et al., Cancer Res., 50(19):6371-6378, 1990.
Ghosh and Bachhawat, In: Liver Diseases, Targeted Diagnosis and Therapy Using Specific Receptors and Ligands, Wu et al. (Eds.), Marcel Delker, N.Y., 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Goodson, In: Medical Applications of Controlled Release, supra, 2:115-138, 1984.
Goossens et al., Int. Immunol., 7:797-805, 1995.
Gopal, Mol. Cell Biol., 5:1188-1190, 1985.
Gossen and Bujard, Proc Natl Acad Sci USA, 89(12):5547-5551, 1992.
Gossen et al., Science, 268(5218):1766-1769, 1995.
Graham and Van Der Eb, Virology, 52:456-467, 1973.
Greene et al., Immunology Today, 10:272, 1989
Grosschedl and Baltimore, Cell, 41:885, 1985.
Harding, Trends Cell Biol., 5:105-109, 1995.
Harland and Weintraub, J. Cell Biol., 101(3):1094-1099, 1985.
Haslinger and Karin, Proc. Natl. Acad. Sci. USA, 82:8572, 1985.
Hauber and Cullen, J Virology, 62:673, 1988.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Howard et al., J. Neurosurg., 71:105, 1989.
Hsieh et al., Science, 260:547-549, 1993.
Huang et al., Cell, 27:245, 1981.
Hug et al., Mol. Cell. Biol., 8:3065, 1988.
Hwang et al., Mol. Cell. Biol., 10:585, 1990.
Ikonomidis et al., Vacc., 15:433-440, 1997.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jensen et al., J. Virol., 71:8467-8474, 1997.
Johnson et al., Mol. Cell. Biol., 9:3393, 1989.
Kadesch and Berg, Mol. Cell. Biol., 6:2593, 1986.
Kaneda et al., Science, 243:375-378, 1989.
Karin et al., Mol. Cell. Biol., 7:606, 1987.
Karin et al., Mol. Cell. Biol., 7:606, 1987.
Katinka et al., Cell, 20:393, 1980.
Kaufmann, Annu. Rev. Immunol., 11: 129-163, 1993.
Kawamoto et al., Mol. Cell. Biol., 8:267, 1988.
Kiledjian et al., Mol. Cell. Biol., 8:145, 1988.
Klamut et al., Mol. Cell. Biol., 10:193, 1990.
Koch et al., Mol. Cell. Biol., 9:303, 1989.
Kraus et al. FEBS Lett., 428(3):165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Kyte and Doolittle, J. Mol. Biol., 157(1):105-132, 1982.
Lam et al., Pro. Int'l. Symp. Control. Rel. Bioact. Mater., 24:759-760, 1997.
Langer, Science, 249:1527-1533, 1990.
Lareyre et al., J. Biol. Ceinem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., Biochem. Biophys. Res. Commun., 238(2):462-467, 1997.
Lee et al., Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Levy et al., Science, 228:190, 1985.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Maniatis, et al., Molecular Cloning, A Laboratory Manual, Cold Spring Harbor Press, Cold Spring Harbor, N.Y., 1990.
Martins et al., Am. J. Tropical Med. Hygiene, 69:292(Abs.230), 2003.
McNeall et al., Gene, 76:81, 1989.
Medical Applications of Controlled Release, Langer and Wise (Eds.), CRC Pres., Boca Raton, Fla., 1974.
Merrifield, Science, 232(4748):341-347, 1986.
Michel et al., Mol. Microbiol., 4:2167-2178, 1990.
Miksicek et al., Cell, 46:203, 1986.
Mordacq and Linzer, Genes and Dev., 3:760, 1989.
Moreau et al., Nucl. Acids Res., 9:6047, 1981.
Muesing et al., Cell, 48:691, 1987.
Ng et al., Nuc. Acids Res., 17:601, 1989.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Ning et al., Radiotherapy & Oncology, 39:179-189, 1996.
Nomoto et al., Gene, 236(2):259-271, 1999.
Ondek et al., EMBO J., 6:1017, 1987.
Ornitz et al, Mol. Cell. Biol., 7:3466, 1987.
Pabniter et al., Nature, 300:611, 1982.
Pan et al., Cancer Res., 55:4776-4779, 1995a.
Pan et al., Cancer Res., 59:5264-5269, 1999.
Pan et al., Nature Med., 1:471-477, 1995b.
PCT Appln. WO 91/05548
PCT Appln. WO 96/20698
PCT Appln. WO 99/15154
PCT Appln. WO 99/20253
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Poffer et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Ranger and Peppas, J. Macromol. Sci. Rev. Macropol. Chem., 23:61, 1983.
Redondo et al., Science, 247:1225, 1990.
Reed and Campos-Neto, Curr. Opin. Immunol., 15(4):456-460, 2003.
Reisman and Rotter, Mol. Cell. Biol., 9:3571, 1989.
Remington's Pharmaceutical Sciences, 18th Ed. Mack Printing Company, 1289-1329, 1990.
Resendez Jr. et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe, et al., Mol. Cell. Biol., 10:689-695, 1990.
Rittling et al., Nuc. Acids Res., 17:1619, 1989.
Rosen et al., Cell, 41:813, 1988.
Safley et al., J. Immunol., 146:3604-3616, 1991.
Sakai et al., Genes and Dev., 2:1144, 1988.
Sambrook et al., In: Molecular cloning, Cold Spring Harbor Laboratory Press, Cold Spring Harbor, N.Y., 2001.
Sanderson et al., J. Exp. Med., 182:1751-1757, 1995.
Satake et al., J Virology, 62:970, 1988.
Schafer et al., J. Immunol., 149:53-59, 1992.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J, 6:1913, 1987.
Shen et al., Proc. Natl. Acad. Sci. USA, 92:3987-3991, 1995.
Sherman et al., Mol. Cell. Biol., 9:50, 1989.
Sleigh and Lockett, J. EMBO, 4:3831, 1985.
Song et al., 1995, PDA J. Pharm. Sci. Tech., 50:372-397, 1995.
Soussi et al., Vacc., 20:2702-2712, 2002.
Spalholz et al., Cell, 42:183, 1985.
Spandau and Lee, J. Virology, 62:427, 1988.
Spandidos and Wilkie, EMBO J, 2:1193, 1983.
Starks et al., J. Immunol., 173:420-427, 2004.
Stephens and Hentschel, Biochem. J, 248:1, 1987.
Stewart and Young, In: Solid Phase Peptide Synthesis, 2^{nd} Ed., Pierce Chemical Co., 1984.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol., 10:165, 1990a.
Taylor and Kingston, Mol. Cell. Biol., 10: 176, 1990b.
Taylor et al., J. Biol. Chem., 264:15160, 1989.
Temin, In.: Gene Transfer, Kucherlapati (Ed.), NY, Plenum Press, 149-188, 1986.
Thiesen et al., J. Virology, 62:614, 1988.
Treisman, Cell, 42:889, 1985.
Tronche et al., Mol. Biol. Med., 7:173, 1990.
Trudel and Constantini, Genes and Dev., 6:954, 1987.
Tsumaki et al., J. Biol. Chem., 273(36):22861-22864, 1998.
Tur-Kaspa et al., Mol. Cell. Biol, 6:716-718, 1986.
Tyndell et al., Nuc. Acids. Res., 9:6231, 1981.
Vannice and Levinson, J. Virology, 62:1305, 1988.
Vasseur et al., Proc Natl. Acad. Sci. USA, 77:1068, 1980.
Wang and Calame, Cell, 47:241, 1986.
Weber et al., Cell, 36:983, 1984.
Weinberger et al. Mol. Cell. Biol., 8:988, 1984.
Wilson et al., Infect. Immun., 63(5):2062-2069, 1995.
Winoto and Baltimore, Cell, 59:649, 1989.
Wu and Wu, Biochemistry, 27: 887-892, 1988.
Wu and Wu, J. Biol. Chem., 262:4429-4432, 1987.
Wu et al., Biochemn. Biophys. Res. Commun., 233(1):221-226, 1997.
Yang et al., Proc. Natl. Acad. Sci. USA, 87:4144-4148, 1990.
Yutzey et al. Mol. Cell. Biol., 9:1397, 1989.
Zhao-Emonet et al., Biochim. Biophys. Acta, 1442(2-3):109-119, 1998.

What is claimed is:

1. A vaccine comprising one or more of the antigens selected from the group consisting of the Leishmania chagasi homologue of L. infantum hypothetical protein and the L. infantum K-39 group protein.

2. The vaccine of claim 1, further comprising both the Leishmania chagasi homologue of L. infantum hypothetical protein and the L. infantum K-39 group protein.

3. The vaccine of claim 1, comprising the Leishmania chagasi homologue of L. infantum hypothetical protein.

4. The vaccine of claim 1, comprising the L. infantum K-39 group protein.

5. The vaccine of claim 1, further comprising one more of the antigens selected from the group Leishmania chagasi homologue of L. major transitional ER ATPase, the Leishmania chagasi homologue of L. infantum glutamine synthetase, the Leishmania chagasi homologue of Leishmania infantum EF-1γ, the Leishmania chagasi homologue of Leishmania infantum A2, and Leishmania chagasi Lcr-1.

6. The vaccine of claim 1, formulated in a lipid delivery vehicle.

7. The vaccine of claim 6, wherein the lipid delivery vehicle is a liposome.

8. The vaccine of claim 1, further comprising an adjuvant.

9. A vaccine comprising a first expression cassette comprising a nucleic acid segment encoding for an antigen selected from the group consisting of the Leishmania chagasi homologue of L. infantum hypothetical protein and L. infantum K-39 group protein.

10. The vaccine of claim 9, wherein said first expression cassette further comprises a nucleic acid segment encoding both the Leishmania chagasi homologue of L. infantum hypothetical protein and the L. infantum K-39 group protein.

11. The vaccine of claim 9, said vaccine further comprising a second expression cassette, where said first expression cassette comprises a nucleic acid segment encoding the Leishmania chagasi homologue of L. infantum hypothetical protein, and said second expression cassette comprises a nucleic acid segment encoding the L. infantum K-39 group protein.

12. The vaccine of claim 9, wherein said first expression cassette comprises a nucleic acid segment encoding the Leishmania chagasi homologue of L. infantum hypothetical protein.

13. The vaccine of claim 9, wherein said first expression cassette comprises a nucleic acid segment encoding the L. infantum K-39 group protein.

14. The vaccine of claim 9, wherein said first expression cassette further comprises a nucleic acid segment encoding comprising one more of the antigens selected from the group Leishmania chagasi homologue of L. major transitional ER ATPase, the Leishmania chagasi homologue of L. infantum glutamine synthetase, the Leishmania chagasi homologue of Leishmania infantum EF-1γ, the Leishmania chagasi homologue of Leishmania infantum A2, and Leishmania chagasi Lcr-1.

15. The vaccine of claim 9, wherein said expression cassette is comprised within a replicable expression construct.

16. The vaccine of claim 15, wherein said replicable expression construct is a viral vector.

17. The vaccine of claim 15, wherein said replicable expression construct is a non-viral vector.

18. The vaccine of claim 15, wherein said replicable expression construct is a Listeria monocytogenes vector.

19. The vaccine of claim 18, wherein said Listeria monocytogenes vector is avirulent.

20. The vaccine of claim 19, wherein said avirulent Listeria monocytogenes vector is ActA⁻¹.

21. The vaccine of claim 17, wherein said non-viral vector is naked DNA.

22. The vaccine of claim 17, wherein said non-viral vector formulated in a lipid delivery vehicle.

23. The vaccine of claim 22, wherein the lipid delivery vehicle is a liposome.

24. A method of inducing an immune response in a subject comprising providing to said subject one or more of:
(a) a vaccine comprising one or more of the antigens selected from the group consisting of the Leishmania chagasi homologue of L. infantum hypothetical protein and the L. infantum K-39 group protein;
(b) a vaccine comprising a first expression cassette comprising a nucleic acid segment encoding for an antigen selected from the group consisting of the Leishmania chagasi homologue of L. infantum hypothetical protein and L. infantum K-39 group protein; and/or
(c) a vaccine comprising an avirulent Listeria monocytogenes vector comprising a nucleic acid segment encoding for an antigen selected from the group consisting of the Leishmania chagasi homologue of L. infantum hypothetical protein and L. infantum K-39 group protein.

25. The method of claim 24, wherein the vaccine comprises one or more of the antigens selected from the group consisting of the Leishmania chagasi homologue of L. infantum hypothetical protein and the L. infantum K-39 group protein.

26. The method of claim 24, wherein the vaccine comprises a first expression cassette comprising a nucleic acid segment encoding for an antigen selected from the group consisting of the Leishmania chagasi homologue of L. infantum hypothetical protein and L. infantum K-39 group protein.

27. The method of claim 24, wherein the vaccine comprises an avirulent Listeria monocytogenes vector comprising a nucleic acid segment encoding for an antigen selected from the group consisting of the Leishmania chagasi homologue of L. infantum hypothetical protein and L. infantum K-39 group protein.

28. The method of claim 24, comprising administering (a) and (b), (a) and (c), (b) and (c), or (a), (b) and (c) to said subject.

29. The method of claim 24, comprising two or more administrations of the same vaccine.

30. The method of claim 24, wherein the vaccine further comprises one more of the antigens selected from the group Leishmania chagasi homologue of L. major transitional ER ATPase, the Leishmania chagasi homologue of L. infantum glutamine synthetase, the Leishmania chagasi homologue of Leishmania infantum EF-1γ, the Leishmania chagasi homologue of Leishmania infantum A2, and Leishmania chagasi Lcr-1 or a nucleic acid segment coding therefor.
